(19) United States
(12) Patent Application Publication
(10) Pub. No.: US 2017/0328814 A1
(43) Pub. Date: Nov. 16, 2017

(54) APPARATUS CONNECTING A WATER SAMPLE BOTTLE TO AN UNMANNED AERIAL VEHICLE (UAV) IN ORDER TO COLLECT WATER SAMPLES FROM BELOW THE SURFACE OF A WATER BODY

(71) Applicant: Hatch Ltd., Mississauga (CA)

(72) Inventors: Devin Noel Castendyk, Denver, CO (US); Brian Straight, Boulder, CO (US); Pierre Filiatreault, Mississauga, CA (US)

(21) Appl. No.: 15/596,713

(22) Filed: May 16, 2017

Related U.S. Application Data
(60) Provisional application No. 62/337,180, filed on May 16, 2016.

(51) Int. Cl. G01N 1/12; G01N 33/18; B64D 1/22; B64C 39/02; G01N 1/10

(52) U.S. Cl. CPC G01N 1/12; B64D 1/22; B64C 39/024; G01N 33/18; B64C 2201/128; G01N 2001/1031

(57) ABSTRACT

An apparatus to connect a multi-parameter probe or water sampling vessel to an Unmanned Aerial Vehicle (UAV), or aerial drone, facilitates the safe collection of samples from various depths in any water body or storage tank. Aspects of the present invention reduce risks to humans, who would, under normal circumstances, be required to be present on the water body surface to carry out sampling. The invention also reduces sampling costs.

### CROSS-REFERENCE TO RELATED APPLICATIONS

The present application claims priority to U.S. Provisional Patent Application No. 62/337,180, filed May 16, 2016,

### FIELD

The present application relates generally to water sampling and, more specifically, to an apparatus connecting a water sample bottle to an unmanned aerial vehicle, in order to collect water samples from below the surface of a water body.

### BACKGROUND

Water bodies and open storage tanks containing fluids often require collection of water samples as part of monitoring programs. Such water quality monitoring usually involves employing a boat and a trained boat crew. In one instance, the boat crew may double as a trained sampling team. In another instance, a trained sampling team may be on-board in addition to the trained boat crew. It is expected that the trained boat crew and sampling team implement numerous safety measures as these working environments are known to have several associated safety risks. This safety component is known to make the sampling aspect of water quality monitoring expensive.

### BRIEF DESCRIPTION OF THE DRAWINGS

Reference will now be made, by way of example, to the accompanying drawings which show example implementations; and in which:

FIG. 1 illustrates an example of an off-the-shelf water sample bottle (e.g., a Niskin bottle) in an open condition;

FIG. 2 illustrates the example water sample bottle of FIG. 1 in a closed condition;

FIG. 3 illustrates an example of the off-the-shelf, unmanned aerial vehicle (UAV) (or “drone”) connected, by way of a tether connected to the UAV by a connection apparatus, to the water sample bottle of FIG. 1 in accordance with aspects of the present application; and

FIG. 4 illustrates, in a bottom plan view, the connection apparatus connecting the UAV to the tether.

### DETAILED DESCRIPTION

Aspects of the present invention relate to an attachment apparatus to connect a liquid sampling bottle to an UAV or drone aircraft adapted for carrying the sampling bottle. Such an attachment apparatus facilitates safe collection of samples from various depths in mine pit lakes and other bodies of liquids and storage tanks. Aspects of the present invention reduce risks to humans, who would, under normal circumstances, be required to be present in a boat on the water surface to carry out the sampling.

The attachment apparatus may include two retractable pistons connected to two independent motors which are remotely activated by a remote controller. One piston holds a static tether adapted to connect to either a multi-parameter probe or a liquid sampling vessel. This piston serves to connect the probe or sample bottle to the UAV and also provides an emergency release mechanism in the event of an entanglement or other unforeseen event. The second piston connects to a lanyard attached to a weighted messenger. Retraction of the second piston causes the messenger to travel down the static tether and close the water sample bottle at the desired water sample depth.

According to an aspect of the present disclosure, there is provided an attachment apparatus for connecting an unmanned aerial vehicle to a tether adapted to connect, at a distal end of the tether, to a liquid sampling vessel or multi-parameter probe, the tether being associated with a messenger adapted to travel along the tether and a lanyard connected, at a distal end of the lanyard, to the messenger. The apparatus includes a primary retractable piston adapted to maintain a primary releasable connection to a proximal end of the tether, a secondary retractable piston adapted to maintain a secondary releasable connection to a proximal lanyard end of the lanyard, a primary piston motor adapted to receive a command to activate and, responsive to receiving the command, release the primary releasable connection, thereby releasing the tether in the event of line entanglement or other emergency thereby protecting the UAV, and a secondary piston motor adapted to receive a command to activate and, responsive to receiving the command, release the secondary releasable connection, thereby releasing the messenger, thereby allowing the messenger to travel along the tether and, upon arrival at the liquid sampling bottle, trigger closure of the liquid sampling bottle.

According to another aspect of the present disclosure, there is provided a method of controlling a sampling event. The method includes receiving a lanyard release command and, responsive to the release command, controlling an apparatus to release a connection between a lanyard and an aircraft attachment, thereby allowing a messenger, connected to the lanyard, to, under influence of gravity, travel along a tether to contact a trigger shaft to initiate the sampling event.

According to a further aspect of the present disclosure, there is provided a method of physiochemical profiling. The method includes following a predetermined path from an origin point to a sampling location, lowering a multi-parameter data sonde a full depth of a water column and returning the multi-parameter sonde to the origin point.

According to an even further aspect of the present disclosure, there is provided an apparatus for connecting an unmanned aerial vehicle to a tether adapted to connect to, at a distal end of the tether, a liquid sampling vessel, the tether being associated with a messenger adapted to travel along the tether and a lanyard connected, at a distal end of the lanyard, to the messenger. The apparatus includes a lanyard release piston adapted to maintain a connection to a proximal end of the lanyard and a lanyard release motor adapted, upon activation, to turn a lanyard release arm through an arc, thereby retracting the lanyard release piston, thereby releasing the connection to the proximal end of the lanyard, thereby allowing the messenger to travel along the tether and, upon arrival at the liquid sampling bottle, trigger closure of the liquid sampling bottle.

Other aspects and features of the present disclosure will become apparent to those of ordinary skill in the art upon review of the following description of specific implementations of the disclosure in conjunction with the accompanying figures.

Collecting samples from a boat may be seen to involve a number of components. The components include a boat, a boat pilot, a back-up boat in case of engine failure, a dock to access the boat (sometimes in the presence of a soft or crumbling shoreline), an access road, access road maintenance, personal floatation devices and crew to be trained in boat safety. Such a collection of components is known to be employed in the act of collecting samples. However, such a collection of components is also known to be relatively expensive. Furthermore, performing the task of collecting samples with such a collection of components is known to be associated with several risks to human health. Such risks may include drowning, asphyxiation from degassing lake water or injury from slope failure or falling rock.

The task of collecting water samples may, alternatively, be accomplished from the skid of a helicopter. Performing the task of collecting water samples in such a manner may involve a person standing on a skid while the associated helicopter is in flight, maintaining a certain altitude above the water body. Accordingly, performing the task of collecting water samples from the skid of a helicopter is known to be associated with several risks to human health. This arrangement for carrying out the task of collecting water samples is also known to be relatively expensive. As such, the arrangement is rarely employed.

One example of a location in which this equipment may be used is a mine pit lake. This is a surface, open pit mine used in metal, coal, diamond, oil sands, and aggregate mining districts which floods with water following the cessation of mining activities. A mining company may, in recognition of the expense and safety risks associated with known methods and arrangements for collecting water samples, opt out of ongoing water quality monitoring of their pit lakes. However, such a course of inaction may be seen to place the mining company out of compliance with industry regulators. Furthermore, without ongoing data related to pit lake water quality, the mining company may be seen as unable to assess the success of measures designed to mitigate negative environmental impacts of the mine associated with the pit lake.

It should be clear that an ongoing water quality monitoring effort involves a step of obtaining water samples. To this end, a vessel may be employed for obtaining water samples. A generic vessel for obtaining a water sample may have a substantially rigid body with two end portions with openings for receiving the water sample. Two end plugs may be deployed to close off the openings, thereby entrapping a water sample inside the body.

The best-known vessel of this general type is a vessel known among those skilled in the art as a “Niskin Bottle,” as described in U.S. Pat. Nos. 3,489,012 and 3,815,422. The full disclosure of the two patents is hereby incorporated herein by reference. Various sizes of Niskin bottles are marketed by, among others, General Oceanics of Miami, Fla. Another common vessel is known as a Van Dorn Water Sampler marketed by, among others, KC Denmark, of Silkeborg, Denmark.

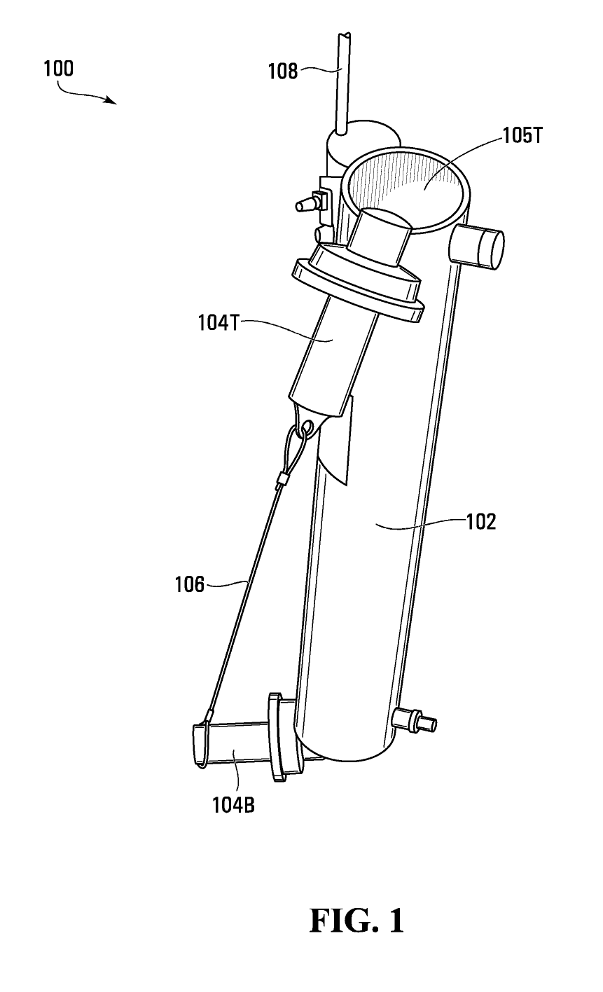

An example Niskin bottle 100 is illustrated in FIG. 1 in an open condition. The Niskin bottle 100 includes a body 102, a top end plug 104T and a bottom end plug 104B. The body 102 has a top opening 105T and a bottom opening (not shown). The top end plug 104T is sized to close off the top opening 105T. Similarly, the bottom end plug 104B is sized to close off the bottom opening.

Notably, the Niskin bottle 100 is illustrated in FIG. 1 as being suspended from a static line tether 108. The tether 108 connects, at a top end, to a sampler-to-aircraft connection apparatus (not shown in FIG. 1) via a retractable piston connector below (not shown in FIG. 1) and, at a bottom end, to the Niskin bottle 100. In one example, the tether 108 is a nylon cord that is 100 m in length.

The top end plug 104T and the bottom end plug 104B are connected in two distinct manners. One connection between the top end plug 104T and the bottom end plug 104B is accomplished on the outside of the body 102 with an outside connector 106. The other connection between the top end plug 104T and the bottom end plug 104B is accomplished on the inside of the body 102 with an inside connector (not shown). The inside connector biases the top end plug 104T towards the bottom end plug 104B inside of the body 102.

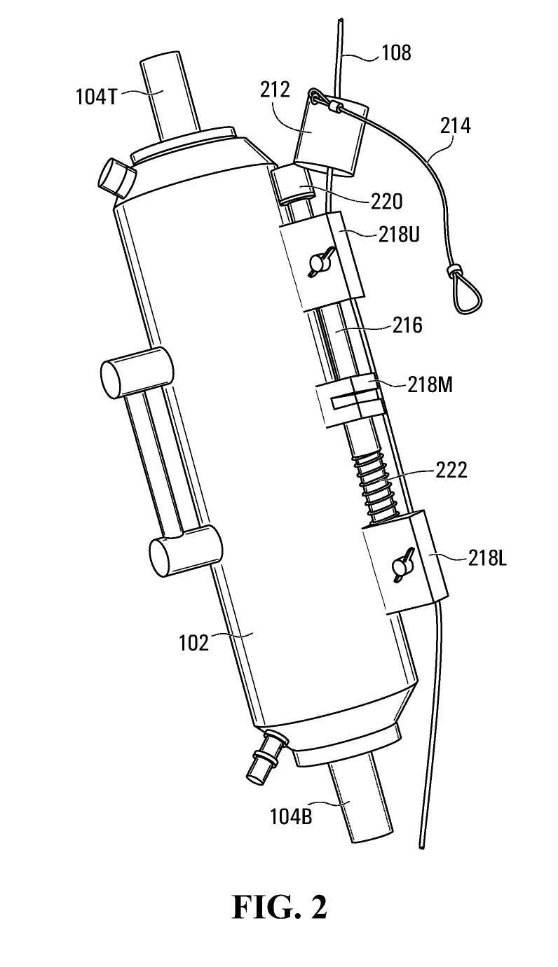

In operation, responsive to a release of the outside connector 106, the Niskin bottle 100 carries out a transition between the open condition illustrated in FIG. 1 to a closed position illustrated in FIG. 2. In the closed position, the top end plug 104T closes off the top opening 105T and the bottom end plug 104B closes off the bottom opening. Responsive to the outside connector 106 being released while the Niskin bottle 100 is under water, a water sample is contained within the body 102.

Several features of the Niskin bottle 100 are more readily reviewed in FIG. 2 than in FIG. 1. For example, a trigger shaft 216 is illustrated in FIG. 2, maintained in a parallel relation with the body 102 by three brackets: an upper bracket 218U; a middle bracket 218M; and a lower bracket 218L. Additionally, the trigger shaft 216 is biased toward the top opening 105T of the body 102 by a biasing element 222. As illustrated in FIG. 2, the biasing element 222 is a spring.

As illustrated in FIG. 2, the tether 108 attaches to the Niskin bottle 100 at the upper bracket 218U. A second piston (not shown in FIG. 2) on the sampler-to-aircraft connector supports a messenger 212 connected to an associated lanyard 214. The messenger 212 may, for example, be implemented as the GO Devil Messenger marketed by General Oceanics of Miami, Fla. The messenger 212 may be cylindrical with, for example, a weight of 1 kg, an outside diameter of 5.1 cm and length of 6.3 cm. For ease of interface with the messenger 212, the trigger shaft 216 has an expanded top end 220.

To move the Niskin bottle 100 into position in a water body for obtaining a water sample, it is proposed herein to employ a rotary-wing aircraft, such as hexi-copter or opti-copter UAV. A rotary-wing aircraft, or “rotorcraft,” is a heavier-than-air flying machine that uses lift generated by wings, called rotary wings or rotor blades, that each revolve around a respective mast.

A multirotor or multicopter is a rotorcraft with more than two rotors. An advantage of multirotor aircraft is simpler rotor mechanics required for flight control. Unlike single- and double-rotor helicopters, which use complex variable pitch rotors, multirotors often use fixed-pitch blades; control of vehicle motion is achieved by varying the relative speed of each rotor to change the thrust and torque produced by each.

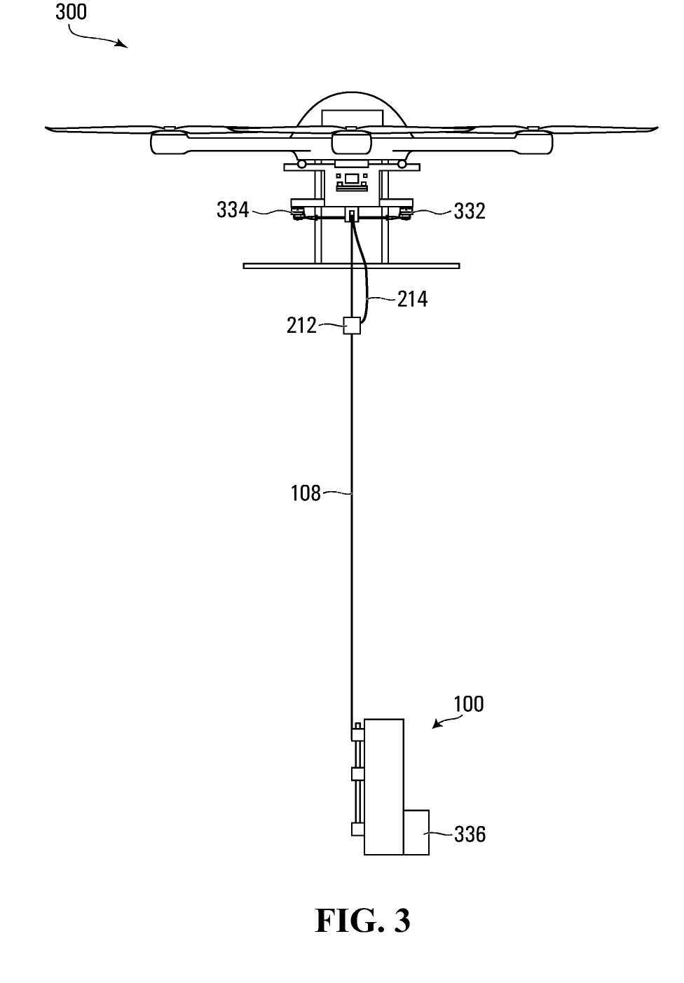

FIG. 3 illustrates the Niskin bottle 100 suspended, by the tether 108, from the UAV 300.

Given a desire to lift 6 kg, it is proposed herein to employ, for the UAV 300, a multicopter marketed under the name “Matrice 600” by DJI of Shenzhen, China. As will be understood, aircraft with distinct lifting capacity may be employed for distinct sizes of water samples. The Niskin bottle 100 may, in one example, have a 1.2 Liter capacity and weigh 3.25 kg when full.

According to aspects of the present application, an attachment 400 developed for the UAV 300 includes a lanyard release 332 and a tether release 334.

According to aspects of the present application, the attachment 400 includes a universal connector so that the attachment 400 may be connected to any UAV capable of supporting the weight load.

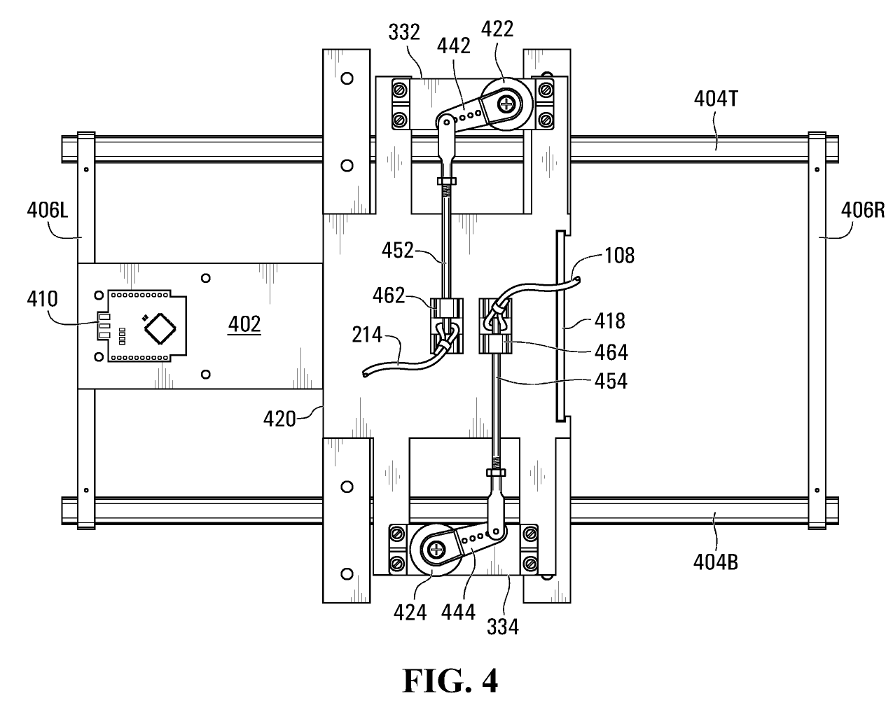

The attachment 400 is illustrated, in bottom plan view, in FIG. 4. The attachment 400 is based on a rectangular frame formed by a top rod 404T and a bottom rod 404B connected, at a left end, by a left connection stage 406L and, at a right end, by a right connection stage 406R. The top rod 404T and the bottom rod 404B support a battery housing 420 inside of which is held a battery (not shown). Access to the battery is provided via a battery cover 418. The lanyard release 332 is mounted to the battery housing 420. Similarly, the tether release 334 is mounted to the battery housing 420.

The lanyard release 332 includes a lanyard release motor 422, a lanyard release arm 442, a lanyard release piston 452 and a lanyard release piston block 462. The lanyard release piston block 462 includes a pair of vanes. Each of the vanes includes an aperture arranged to receive the lanyard release piston 452. In use, a loop in the lanyard 214 receives the lanyard release piston 452 between the vanes of the lanyard release piston block 462. The lanyard release motor 422 may be implemented as a stepper motor and, more specifically, a servo motor.

The tether release 334 includes a tether release motor 424, a tether release arm 444, a tether release piston 454 and tether release piston block 464. The tether release piston block 464 includes a pair of vanes. Each of the vanes includes an aperture arranged to receive the tether release piston 454. In use, a loop in the tether 108 receives the tether release piston 454 between the vanes of the tether release piston block 464. The tether release motor 424 may be implemented as a stepper motor and, more specifically, a servo motor.

The attachment 400 also includes a platform 402 between the battery housing 420 and the left connection stage 406L. The platform 402 supports a processor 410. The processor 410 receives electrical power from the battery and is communicatively connected to the lanyard release motor 422 and the tether release motor 424. The processor 410 may be associated with radio receiver circuitry (not shown).

In operation, a human drone pilot commands the aircraft 300 to carry the Niskin bottle 100, in the open condition as illustrated in FIG. 1, to a particular position over a pit lake. The human drone pilot then commands the aircraft 300 to reduce altitude until the Niskin bottle 100 is in the pit lake at a desired depth. Preferably, the vertical resolution of the aircraft 300 is plus or minus 50 centimeters. The human drone pilot or assistant pilot then arranges transmission of a lanyard release command to the attachment 400 to release the lanyard 214 supporting the messenger 332. The lanyard release command may be relayed by a separate remote controller. Responsive to receiving the lanyard release command, the processor 410 activates the lanyard release motor 422. Responsive to activation, the lanyard release motor 422 causes the lanyard release arm 442 to turn through an arc, thereby retracting the lanyard release piston 452 from between the vanes of the lanyard release piston block 462. With the lanyard release piston 452 absent from between the vanes of the lanyard release piston block 462, the lanyard 214 is released, thereby allowing the messenger 212 (with the lanyard 214) to, under the influence of gravity, slide down the tether 108, toward the Niskin bottle 100. Upon arriving at the Niskin bottle 100 at the desired depth in the pit lake, the messenger 212 contacts the top end 220 of the trigger shaft 216. Responsive to contact from the messenger 212, the trigger shaft 216 acts to release the outside connector 106. As discussed hereinbefore, upon release of the outside connector 106 and due to the biasing of the inside connector, the top end plug 104T closes off the top opening 105T and the bottom end plug 104B closes off the bottom opening. That is, the Niskin bottle 100 goes through a transition into the closed condition illustrated in FIG. 2.

The human drone pilot then commands the aircraft 300 to increase its altitude, such that the closed Niskin bottle 100 is extracted from the lake. Subsequently, the human drone pilot commands the aircraft 300 to return to a home base position, perhaps in the vicinity of the human drone pilot. It is contemplated that it would be preferable to maintain a spigot at the bottom of the Niskin bottle 100 out of contact with dirt and sand at the home base location. To that end, there may be a Niskin bottle cradle (not shown) into which the human drone pilot may guide the aircraft 300 to place the Niskin bottle 100 before the human drone pilot commands the aircraft 300 to land. The Niskin bottle cradle may, for example, be cylindrical with dimensions larger than the dimensions of the Niskin bottle 100, so that the Niskin bottle 100 may be easily received by the Niskin bottle cradle.

It is contemplated that the floor of the pit lake may not always be known and there may be instances wherein the Niskin bottle 100 becomes stuck in the pit lake. To avoid damage to the aircraft 300, which may have a value orders of magnitude higher than the value of the Niskin bottle 100, the aircraft 300 includes the tether release 334. In an instance wherein the Niskin bottle 100 becomes stuck, the human drone pilot may decide to command the attachment 400 to release the tether 108, thus releasing the water sample bottle 100. Responsive to receiving the tether release command, the processor 410 activates the tether release motor 424. Responsive to activation, the tether release motor 424 causes the tether release arm 444 to turn through an arc, thereby retracting the tether release piston 454 from between the vanes of the tether release piston block 464. With the tether release piston 454 absent from between the vanes of the tether release piston block 464, the tether 108 is released, thereby disconnecting the aircraft 300 from the stuck Niskin bottle 100.

If the condition of the stuck Niskin bottle 100 occurs when the lanyard 214 (and, accordingly, the messenger 212) remain connected to the aircraft, the human drone pilot may also arrange transmission of a lanyard release command to the attachment 400.

As illustrated in FIG. 3, the Niskin bottle 100 may have, attached thereto, optional equipment 336. The optional equipment 336 may include: equipment for in situ measurement of conductivity of the water in the pit lake; equipment for in situ measurement of temperature of the water in the pit lake; equipment for in situ measurement of density of the water in the pit lake; a depth sounder; equipment for in situ measurement of pH of the water in the pit lake; equipment for in situ measurement of Dissolved Oxygen of the water in the pit lake; equipment for in situ measurement of turbidity of the water in the pit lake; and a pressure transducer for in situ measurement of pressure, thereby providing a redundant indication of the depth at which as particular sample has been captured.

Pit lakes have been described hereinbefore as resultant from open pit mining. It should be clear that pit lakes may also be associated with other forms of mining. For example, the effort to extract oil sands is not called open pit mining, but does lead to pit lakes. Pit lakes are also associated with diamond mining and coal mining. Extraction of aggregate in quarries may also be seen to lead to pit lakes.

Bodies of water that are not specifically pit lakes may also be subject to testing using the apparatus described herein. For example, tailings ponds used to receive mill tailings at mine sites, evaporation ponds used in the potation, lithium and natural gas industries, and a municipal drinking water reservoir may be candidates for such testing. For another example, open tanks of water or process water found at a waste water treatment plants or Alumina processing facilities may be subject to testing to monitor, for instance, nitrogen levels and to determine the extent to which solids removal has been successful.

Through the foregoing description, it has been discussed that water is being sampled. It should be clear that liquids other than water may also be subject sampling. Cucumber pickle factories usually ferment cucumbers in large outdoor vats of salt brine. These vats typically have no cover and benefit from the sun's ultraviolet light to prevent yeast and mold growth on the brine surface. Accordingly, such open vats are suitable candidates for drone-based sampling.

It is known to dispose of produced water, a byproduct of oil well and gas well operation, in large evaporation ponds.

When lithium salts are extracted from the water of mineral springs, brine pools and brine deposits, the lithium salts are separated from other elements by pumping the lithium-rich brine into solar evaporation ponds.

In the known potash solution mining process, water containing dissolved potash is pumped out of a cavern to the surface, where the water may be evaporated in solar evaporation ponds, leaving behind salt and potash.

It should be clear that such evaporation ponds are suitable candidates for drone-based sampling.

Mining operations are often associated with ponds of “tailings.” In particular, tailings ponds may be associated with gold mining operations as well as coal mining operations. It should be clear that such tailing ponds are suitable candidates for drone-based sampling.

In addition to the mining industry, liquids are also used in processing metals. For example, open processing tanks are often employed in the aluminum industry. It should be clear that such open processing tanks are suitable candidates for drone-based sampling.

To this point, the liquids that have been discussed as suitable for testing have, largely, been fresh-water-based liquids. It should be clear that sampling according to aspects of the present application may also include salt-water sampling. For example, in the event of an off-shore oil spill, a combination of aircraft 300, the sampler-to-aircraft connection apparatus 108 and sampler 100 may be employed to obtain samples of the surface of the ocean.

In the foregoing, the sampler-to-aircraft connection apparatus 108 has been described as having a fixed length. Accordingly, placing the Niskin bottle at a particular depth within a pit lake involves appropriately altering the altitude of the aircraft 300. Optionally, a winch (not shown) may be fixed to the tether release 334. In this case, the aircraft may maintain a constant altitude while the winch is commanded to wind out the sampler-to-aircraft connection apparatus 108 such that the Niskin bottle is arranged to achieve the particular depth. The winch may be, for example, controlled using commands to the aircraft 300 related to gimbal control. Alternatively, the winch may include a capability to receive commands wirelessly. While a winch may be used with nylon cord, it is contemplated that by using a strong, thin, light metal cable for the sampler-to-aircraft connection apparatus 108, compatibility with the winch may be improved.

In the foregoing, samples are generally collected using the Niskin bottle 100. It is further contemplated that a sampling bottle with a custom design may be employed. The custom bottle may, for example, be formed from carbon fiber such that weight is optimized. Recall that a given multicopter has a particular payload capacity and that a Niskin bottle is not, generally, designed to be flown. Accordingly, the Niskin bottle has not necessarily been weight-optimized. By optimizing the weight of the bottle 100, more weight can be apportioned to other aspects.

Rather than, or in addition to, collecting samples and delivering the samples to a laboratory for analysis, the apparatus (the combination of the sampler bottle 100, the aircraft 300 and the sampler-to-aircraft connection apparatus 108) may be configured for real-time monitoring and reporting. A manager of an evaporation pond may, for example, wish to monitor electrical conductivity.

While the apparatus has, to this point, been discussed as having real-time control by one or more operators, it is further contemplated that an obstacle-free path from an origin point to a sampling location may be established. A routine set of instructions may direct the apparatus to use the pre-determined path to fly from the origin point to the sampling location, lower a multi-parameter data sonde a full depth of a water column and return the multi-parameter data sonde to the origin point without constant supervision. Indeed, an operator using an application (“app”) on a mobile device, such as an iPhone™ or an Android™ device, could use the app to establish the timing (when), location (where) and other details (how deep) for the collection of a sample. Alternatively, a routine set of instructions may direct the apparatus to use the pre-determined path to fly from the origin point to the sampling location, obtain a sample and return to the origin point without constant supervision.

The above-described implementations of the present application are intended to be examples only. Alterations, modifications and variations may be effected to the particular implementations by those skilled in the art without departing from the scope of the application, which is defined by the claims appended hereto.

What is claimed is:

1. An attachment apparatus for connecting an unmanned aerial vehicle to a tether adapted to connect, at a distal end of the tether, to a liquid sampling vessel or multi-parameter probe, the tether being associated with a messenger adapted to travel along the tether and a lanyard connected, at a distal end of the lanyard, to the messenger, the apparatus comprising:
a primary retractable piston adapted to maintain a primary releasable connection to a proximal end of the tether;
a secondary retractable piston adapted to maintain a secondary releasable connection to a proximal lanyard end of the lanyard;
a primary piston motor adapted to receive a command to activate and, responsive to receiving the command, release the primary releasable connection, thereby releasing the tether in the event of line entanglement or other emergency thereby protecting the UAV; and
a secondary piston motor adapted to receive a command to activate and, responsive to receiving the command, release the secondary releasable connection, thereby releasing the messenger, thereby allowing the messenger to travel along the tether and, upon arrival at the liquid sampling bottle, trigger closure of the liquid sampling bottle.

2. The attachment apparatus of claim 1 further comprising radio receiver circuitry.

3. The apparatus of claim 1 wherein the liquid sampling vessel comprises a water sampling vessel.

4. A method of controlling a sampling event, the method comprising:
receiving a lanyard release command; and
responsive to the release command, controlling an apparatus to release a connection between a lanyard and an aircraft attachment, thereby allowing a messenger, connected to the lanyard, to, under influence of gravity, travel along a tether to contact a trigger shaft to initiate the sampling event.

5. The method of claim 4 wherein the trigger shaft is associated with a sampling bottle in a liquid to be sampled in a sampling location.

6. The method of claim 5 wherein the liquid to be sampled comprises water.

7. The method of claim 5 wherein the sampling location comprises a mine pit lake; an evaporation pond; a tailings pond; or an open processing tank.

8. The method of claim 7 wherein the pit lake is associated with:
a metal mining operation;
a diamond mining operation; or
a coal mining operation.

9. The method of claim 5 wherein the sampling location comprises a processing tank at an aluminum processing facility.

10. The method of claim 5 wherein the sampling location comprises an evaporation pond.

11. The method of claim 10 wherein the evaporation pond is associated with:
a lithium mining operation;
a potash mining operation; or
a natural gas extraction operation.

12. The method of claim 5 wherein the liquid to be sampled comprises brine.

13. The method of claim 5 wherein the sampling location comprises a drinking water reservoir or a waste water treatment holding tank.

14. A method of physiochemical profiling comprising:
following a predetermined path from an origin point to a sampling location;
lowering a multi-parameter data sonde a full depth of a water column; and
returning the multi-parameter sonde to the origin point.

15. The method of claim 14 wherein the sampling location comprises: a mine pit lake; an evaporation pond; a tailings pond; or an open processing tank.

16. The method of claim 15 wherein the mine pit lake is associated with:
a metal mining operation;
a diamond mining operation; or
a coal mining operation.

17. The method of claim 14 wherein the sampling location comprises a processing tank at an aluminum processing facility.

18. The method of claim 14 wherein the sampling location comprises an evaporation pond.

19. The method of claim 18 wherein the evaporation pond is associated with
a lithium mining operation;
a potash mining operation; or
a natural gas extraction operation.

20. The method of claim 14 wherein the sampling location comprises a drinking water reservoir or a waste water treatment holding tank.

21. An apparatus for connecting an unmanned aerial vehicle to a tether adapted to connect to, at a distal end of the tether, a liquid sampling vessel, the tether being associated with a messenger adapted to travel along the tether and a lanyard connected, at a distal end of the lanyard, to the messenger, the apparatus comprising:
a lanyard release piston adapted to maintain a connection to a proximal end of the lanyard; and
a lanyard release motor adapted, upon activation, to turn a lanyard release arm through an arc, thereby retracting the lanyard release piston, thereby releasing the connection to the proximal end of the lanyard, thereby allowing the messenger to travel along the tether and, upon arrival at the liquid sampling bottle, trigger closure of the liquid sampling bottle.
